(19) United States
(12) Patent Application Publication
(10) Pub. No.: US 2026/0243724 A1
(43) Pub. Date: Aug. 20, 2026

(54) FIELD-EFFECT TRANSISTOR BIOSENSORS INTEGRATED WITH POROUS SENSING MEMBRANE

(71) Applicants: THE UNIVERSITY OF CHICAGO, Chicago, IL (US); THE REGENTS OF THE UNIVERSITY OF CALIFORNIA, Oakland, CA (US)

(72) Inventors: Hyun-June Jang, Lombard, IL (US); Hyouarm Joung, Los Angeles, CA (US); Aydogan Ozcan, Los Angeles, CA (US); Junhong Chen, Chicago, IL (US)

(73) Assignees: THE UNIVERSITY OF CHICAGO, Chicago, IL (US); THE REGENTS OF THE UNIVERSITY OF CALIFORNIA, Los Angeles, CA (US)

(21) Appl. No.: 19/152,406

(22) PCT Filed: Mar. 5, 2024
(86) PCT No.: PCT/US2024/018551

§ 371 Date: Jul. 30, 2025

Related U.S. Application Data
(60) Provisional application No. 63/488,722, filed on Mar. 6, 2023.

(51) Int. Cl. G01N 27/414; C12Q 1/00; G01N 27/327; G01N 33/92

(52) U.S. Cl. CPC G01N 27/4145; C12Q 1/002; G01N 27/3278; G01N 27/4146; G01N 33/92

(57) ABSTRACT

A FET-based biosensor utilizes a dry chemistry approach to achieve sensitive, selective, and miniaturized cost-effective biosensing by integrating two heterogeneous sensing platforms that include electrochemical paper-based assays and electronic field-effect transistor (FET) transducers. The FET-based biosensor eliminates sample matrix effects and the need for wet surface chemistry to functionalize probes on the sensing surface of FET-based biosensors. The biosensor was used for cholesterol detection in clinical plasma samples with a measurement range of 0 to 299 mg/dL using a custom-designed cartridge connected to a FET readout system. The FET readout system detected protons resulting from enzymatic reactions to lipoproteins within a porous sensing membrane impregnated with dry reagents. The results showed an accuracy of 99.4% compared to a commercial assay, and a maximum coefficient of variation of <14%. Deep learning can be used to improve the results even further.

### RELATED APPLICATION

This application claims priority to U.S. Provisional Patent Application No. 63/488,722 filed on Mar. 6, 2023 which is hereby incorporated by reference. Priority is claimed pursuant to 35 U.S.C. § 119 and any other applicable statute.

### STATEMENT REGARDING FEDERALLY SPONSORED RESEARCH AND DEVELOPMENT

This invention was made with government support under 1648451 awarded by the National Science Foundation. The government has certain rights in the invention.

### TECHNICAL FIELD

The technical field generally relates to devices, systems, and methods that use field-effect transistors (FETs) for the detection and/or quantification of target species such as biomarkers in body fluids. More specifically, the technical field relates to an electrochemical detection system and device that incorporates a cartridge having porous sensing membrane therein that includes dried reagents and the like that is disposed adjacent to one or more working electrodes that are coupled to respective FETs.

### BACKGROUND

There is a growing need for point-of-care (POC) testing devices that enable users to test and monitor a variety of health conditions outside of a hospital or clinical setting. For example, home-based POC testing devices have several benefits including cost savings, convenience, better risk management, and health equity (the ability to provide care to patients with mobility limitations). Current POC devices, however, mainly serve as supplementary testing functions near a clinical treatment site with limited testing menus: for example, blood gases, electrolytes, cardiac markers, creatinine, diabetes, drug screening, human chorionic gonadotropin, HIV, influenza, COVID-19, and urinalysis. Also, most optical spectroscopies utilized in POC devices are challenging to implement in at-home settings beyond clinics because they are bulky and expensive and require complex logistics and trained experts. On the other hands, electrochemical detection holds promise for future home diagnostic tools. Advantages include miniaturized reading systems, quantification capabilities, rapid and multiplexed detection capacity. Electrochemical sensors typically use a micron-sized working electrode that measures electrochemical signals resulting from redox reactions of targeted molecules. The success of electrochemical biosensors, however, has mostly been noticeable for glucometers mainly because of a lack of sensitivity and selectivity caused by sample matrix effects. Matrix effects include the effects on an analytical assay caused by all other sample components except the specific compound (analyte) to be analyzed.

Meanwhile, electrical signaling methods by FETs have drawn significant attention over the past decades due to advantages of rapid, real-time detection and extremely high sensitivity to diverse biomarkers. However, commercialization of FET-based biosensors has not taken hold. Three main technical hurdles remain such as interference by sample matrix effects caused by non-specific binding signals and biofouling of the working electrode, Debye screening length issues, and complex logistics. Moreover, sensing surfaces need further protection under wet environments to prevent any denaturation of probes functionalized on either the gate or the semiconducting channel of a FET until usage. This type of wet chemistry approach creates numerous potential issues such as short shelf-life, low yield, leaking, contamination, and challenges in packaging. Moreover, it is imperative that the wet chemistry approach incorporates an additional reagent control using microfluidics. Microfluidics, however, often demands expensive, time-consuming and labor-intensive manufacturing processes to form precise microfluidic channels, often suffering from a low yield of fabrication. Microfluidics also typically require ancillary pumping devices that add further cost and complexity to these devices.

### SUMMARY

In one embodiment, a biosensor-based device or system is disclosed that combines one or more field-effect transistors (FETs) with porous sensing membrane to achieve a new diagnostic platform. The FETs can read out electrochemical kinetics of a specific enzyme or other biochemical reaction in real-time which is triggered by target reactions of a specific biomarker or analyte in body fluids such as whole blood, serum, saliva, urine, sweat, and tears which are loaded into a cartridge device. The cartridge device includes a chamber which receives the sample. One or more porous sensing membrane is/are located within the cartridge and is designed for the body fluids to flow through. The porous sensing membrane may incorporate multiple components such as enzymes, enzyme-labeled antibodies, surfactants, substrates, stabilizers, buffers, and other reagents, all of which may exist in a dry form on or within the porous sensing membrane. The enzyme reaction on the porous sensing membrane is specifically triggered by target biomarkers or analytes in the body fluids, which creates either protons or charged molecules or species by electroactive reactions as a product. The porous sensing membrane delivers specific, pure electrochemical signals to one or more working electrodes disposed in the cartridge after filtering undesirable components of the body fluids inside porous sensing membrane. The working electrode(s), which may be made from materials such as indium tin oxide (ITO), tin oxide (SnO₂), zinc oxide (ZnO₂), metal oxide semiconductors, graphene, reduced graphene oxide, conductive polymers such as regioregular poly(3-hexylthiophene-2,5-diyl) (P3HT), poly(3,4-ethylenedioxythiophene) polystyrene sulfonate (PEDOT:PSS) or other conductive 2D nanomaterials such as molybdenum disulfide, is/are remotely connected to a gate of field-effect transistor (FET) (or multiple such FETs) and receives purified electrochemical signals without sample matrix effects. A reference electrode, such as an Ag/AgCl reference electrode, calomel electrode, copper-copper sulfate electrode, palladium-hydrogen electrode, dynamic hydrogen electrode, mercury-mercurous sulfate electrode, graphite, and platinum is used to apply the gate voltage. Thus, the FET transforms the change in electrochemical potentials on the working electrode in terms of threshold voltage (Vₜₕ) or drain current in real-time. Kinetic signals obtained are also utilized for the detection and/or quantification of biomarkers or analytes in body fluids.

In some embodiments, multiple layers of porous sensing membrane and associated working electrodes are coupled to respective FETs which measure the change in electrochemical potentials on the respective working electrodes or drain current so as to perform multiplex testing for multiple biomarkers or target analytes. The FET-based biosensor device provides a cost-effective, multiplexed, real-time monitoring system of diverse biomarkers in body fluids for point-of-care (POC) or self-testing tools. None of the existing enzymatic assays and immunoassays have measured the kinetics of multiple target biomolecules for POC and self-testing setup due to complexity of the optical detection mechanism. Measurements of enzymatic kinetics opens the opportunity to achieve a full lipid profiling system with direct measurements for, for example, low-density lipoprotein cholesterol (LDL-C), high-density lipoprotein cholesterol (HDL-C), and total cholesterol (TC) in finger prick blood. It should be appreciated that other biomarkers may be also detected and/or quantified using the device and system described herein.

As explained herein, in one embodiment, a cartridge-based biosensor device is provided that receives the fluid sample. The cartridge includes porous sensing membrane disposed adjacent to a working electrode and separate via a gap or space that is used with a FET readout system to analyze electroactive enzymatic reactions occurring inside the cartridge. The unique configuration of the porous sensing membrane with the working electrode built inside the cartridge eliminates sample matrix effects and there is no need for microfluidic systems for controlling reagents and arduous wet chemistry processes for functionalizing biomaterials on the FET sensing surface. A proof-of-concept blood test is shown for cholesterol measurements in patient plasma ranging from 0 to 299 mg/dL. Tests were performed with fifty-six (56) cartridges, and the results showed 99.4% correlation with a commercial clinical assay for measuring cholesterol and a maximum coefficient of variation (CV) of less than 14%.

In one embodiment, a biosensor device includes a cartridge having a chamber for receiving a fluid sample and porous sensing membrane disposed in the chamber and comprising one or more dried enzymes, enzyme-labeled antibodies, surfactants, substrates, stabilizers, buffers, or reagents. A working electrode is disposed in the cartridge and spaced apart from a surface of the porous sensing membrane. The working electrode is electrically coupled to the gate electrode of a FET. A reference electrode is configured to contact with the fluid sample located in the cartridge.

In another embodiment, a biosensor device includes a cartridge having a chamber for receiving a fluid sample and a plurality of porous sensing membranes disposed in the chamber, each porous sensing membrane comprising one or more dried enzymes, enzyme-labeled antibodies, surfactants, substrates, stabilizers, buffers, or reagents. A plurality of working electrodes are disposed in the cartridge and spaced apart from respective surfaces of the plurality of porous sensing membrane. Each working electrode is electrically coupled to respective gates of different FETs in a FET readout system.

In another embodiment, a system for the detection and/or analysis of one or more biomarkers or analytes (e.g., target(s)) in a fluid sample includes a cartridge-based biosensor device having a chamber for receiving a fluid sample. The biosensor device includes one or more porous sensing membrane disposed in the chamber, the one or more porous sensing membrane including one or more dried enzymes, enzyme-labeled antibodies, surfactants, substrates, stabilizers, buffers, or reagents. The biosensor device further includes one or more working electrodes disposed in the cartridge and spaced apart from the one or more porous sensing membrane. A reference electrode is configured to contact with the fluid sample in the chamber. The system includes one or more field-effect transistors (FETs) having a source, drain, and gate, wherein the respective gates of the one or more FETs is/are electrically coupled to the one or more working electrodes. A semiconductor parameter analyzer or current-voltage circuitry is coupled to the reference electrode and the one or more FETs.

In another embodiment, a method for the detection and/or analysis of a biomarker or analyte (e.g., target) in a fluid sample includes providing a cartridge-based biosensor device having a chamber for receiving the fluid sample, the biosensor device including one or more porous sensing membrane disposed in the chamber, the one or more porous sensing membrane comprising one or more dried enzymes, enzyme-labeled antibodies, surfactants, substrates, stabilizers, buffers, or reagents. The biosensor device further includes one or more working electrodes disposed in the cartridge and spaced apart from the one or more porous sensing membrane. One or more field-effect transistors (FETs) are electrically coupled at respective gates to the one or more working electrodes. The fluid sample containing the biomarker(s) or analyte(s) is loaded into an inlet located in the cartridge of the biosensor device. A change in initial threshold voltage (Vₜₕ) or initial drain current at constant voltage is measured for the one or more FETs with a semiconductor parameter analyzer or current-voltage circuitry. A concentration of the biomarker or analyte is output based at least in part on the change in initial Vₜₕ or initial drain current for the one or more FETs.

This biosensor device offers several advantages: 1) imposing higher sensitivity to paper analytical devices empowered by electrical measurements, 2) enabling the measurement of kinetic data, 3) eliminating the complexities associated with laborious wet chemistry procedures for functionalizing biomolecules on the FET sensing surface, 4) removing the need for traditional microfluidic systems to regulate reagents, 5) facilitating cost-effective production (~$0.15 per cartridge, 6) mitigating sample matrix effects, 7) achieving easy miniaturization, and 8) ensuring a prolonged shelf-life.

### BRIEF DESCRIPTION OF THE DRAWINGS

FIG. 1 illustrates a biosensor device for biosensing of a biomarker or analyte. The biosensor device may be in the form of a cartridge. The biosensor device has a working electrode coupled to the gate of a FET. A semiconductor parameter analyzer or similar voltage/current circuitry is used as the FET readout device to measure a change in electrical properties of the FET such as transfer curve, mobility, Vₜₕ and transconductance (Gₘ) for the FET.

FIG. 2A illustrates a perspective view of a two-part cartridge that contains the biosensor device. The two-part cartridge includes an upper portion and a lower portion. Sample is loaded into the upper portion and may undergo various sample pre-processing operations (e.g., filtering). The lower portion contains the porous sensing membrane and working media. The upper portion may connect to the lower portion by twisting the upper portion to lock into the lower portion.

FIG. 2B illustrates exploded views of the upper portion and lower portion of the cartridge. The upper portion may include pads, layers, filters, etc. that are used in pre-processing of the sample (e.g., filtering of red blood cells from sample). The lower portion includes the porous sensing membrane as well as the working electrodes.

FIG. 3A illustrates an alternative embodiment for multiplex testing of biomarkers or analytes. The single-use device includes a chamber that contains a plurality of porous sensing membranes (e.g., sensing membranes) along with a plurality of working electrodes. Each porous sensing membrane is paired with a corresponding working electrode in a vertical configuration. A body fluid sample is introduced into the top of the cartridge and interacts with the different porous sensing membrane/working electrode pairs. Fluid flows or progresses vertically downward from top to bottom. Each working electrode is coupled to a separate FET in the FET-based detection system. Each porous sensing membrane/working electrode pair may be used to detect a different biomarker or analyte (e.g., target) for multiplex testing and analysis.

FIG. 3B illustrates the components making of the single-use cartridge according to one embodiment.

FIG. 3C schematically illustrates a cross-sectional view of the assembled single-use cartridge using the components of FIG. 3B.

FIG. 4 schematically illustrates a biosensor device and illustrates buffering/mixing/filtering taking place in the porous sensing membrane. The configuration results in pure signal transmittance to the working electrode.

FIG. 5 illustrates the sequence of enzymatic reactions used to measure cholesterol with the biosensor device.

FIG. 6A illustrates a graph showing initial threshold voltage levels of clinical plasmas with different cholesterol concentrations.

FIG. 6B illustrates coefficient of variation (CV) values of initial Vₜₕ for each case in FIG. 6A.

FIG. 7A schematically illustrates sample introduction into a biosensor device. Enzymatic reactions take place in the porous sensing membrane and protons or other charged species/molecules accumulate in the gap formed above the working electrode.

FIG. 7B illustrates kinetical curves of change in Vₜₕ as a function of time for different concentrations of cholesterol.

FIG. 8 presents a graph illustrating the variation in Vₜₕ across different cholesterol concentrations, alongside the CV % measured from all cartridges within the same batch.

FIGS. 9A-9C illustrate examples of Vₜₕ values as a function of time for normal (FIG. 9A) and error conditions (membrane leaking (FIG. 9B) and combination failure (FIG. 9C)).

FIG. 10A illustrates kinetical data from ELISA results measuring enzymatic reactions that result from the activity of an LDL specific surfactant in the experimental cholesterol solutions.

FIG. 10B illustrates kinetical data obtained from the biosensor device measuring enzymatic reactions that result from activity of an LDL specific surfactant in clinical plasmas on ITO (without porous sensing membrane yet).

FIG. 11 schematically illustrates a biosensor device connected to a FET-based detection system that is used to assay cholesterol levels.

FIG. 12A illustrates a graph illustrating the pH sensitivity of ITO and CV values of E_{ITO} for each pH over 5 different ITO working electrodes.

FIG. 12B illustrates hysteresis tested by a rapid pH loop of pH 7-4-7-10-7 and E_{ITO} response with different volume sizes (20, 40, 100 μL) of each pH solution (pH4, 7, 10) on ITO.

FIG. 12C illustrate the E_{ITO} drift measured in pH7 for 33 min over 5 different ITO working electrodes.

FIG. 13A illustrates E_{ITO} distribution from lipoprotein-deleted and clinical plasma samples on the bare ITO surface measured.

FIG. 13B illustrates initial E_{ITO} distribution given by mixtures between sending solution (SS) and plasma samples with different volumes on the bare ITO surface (before enzyme reaction); SS without mixing plasma was compared at the same plot.

FIG. 13C illustrates ΔE_{ITO} distribution from mixtures between SS and plasma samples with different volumes on the bare ITO (after enzyme reaction); ΔE_{ITO} was obtained at a difference between initial E_{ITO} and E_{ITO} measured after 10 min.

FIG. 14 illustrates a flowchart of operations for using the biosensor device. Optional use of a trained neural network is illustrated.

FIG. 15 illustrates a system that includes the biosensor device. The system includes the biosensor device and the semiconductor parameter analyzer or current-voltage circuitry. A computing device such as Smartphone is illustrated that displays the results to the assay(s) to the user. An optional trained neural network is illustrated which can improve results further.

FIG. 16 illustrates a schematic of the workflow of the deep learning (DL) signal process framework used to output the concentration of the biomarker or analyte.

FIG. 17A depicts representative real-time Vₜₕ curves generated in response to enzyme reactions, correlating with the cholesterol concentrations detected in human plasma as measured from the cartridge.

FIG. 17B illustrates initial Vₜₕ distributions of cartridges dried with different buffer components such as PBS, PIPES/PBS, PIPES. CV values of initial Vₜₕ were compared.

FIG. 17C shows ΔVₜₕ variation of the cartridge with bare ITO and BSA/ITO for the injection of lipoprotein-free plasma. ΔVₜₕ was defined as the difference between the initial Vₜₕ and Vₜₕ for a specific time of each cartridge.

FIG. 17D illustrates LOD evaluation measured by using a diluted clinical plasma sample with lipoprotein-free plasma.

FIG. 17E illustrates ΔVₜₕ distribution of 178 testing cartridges with plasma samples of varying cholesterol concentrations. These measurements are derived from 15 different sub-batch fabrications of the cartridge.

FIG. 17F illustrates CV values of ΔVₜₕ in FIG. 17E calculated from at least 3 repeated tests for the same plasma.

FIG. 18 illustrates the correlation (R² value) was calculated for the range of cholesterol levels from 0 to 300 mg/dL in human plasma vs. enzyme concentrations of the sensing solution (SS). The SS is an enzyme solution including all detection components which is prepared for drying it on porous sensing membrane. This experiment setup is based on wet chemistry approach (i.e., no cartridge). 19 μL SS was first placed on the bare ITO and sequentially 1 μL plasma samples were added on SS for tests. The cholesterol concentrations in plasma samples were measured using Roche Cobas instrumentation. Concentrations of enzymes exceeding 200 U/mL displayed saturated correlations (R² value), leading us to choose 300 U/mL enzyme concentrations for COE, COx, and POx in all subsequent experiments.

FIG. 19A illustrates a schematic of transfer curves changes over different stages of the enzymatic reaction on the cartridge.

FIG. 19B illustrates representative Gₘ variation over the enzymatic reaction.

FIG. 19C illustrates transfer curve heatmaps after subtraction of the first cycle signal.

FIG. 19D illustrates a comparison of cholesterol concentrations predicted by the neural network using the pure signal and raw heatmap.

FIG. 20A illustrates representative transfer curves captured over 343 sec (total 49 measurements) from 200 mg/dL plasma sample.

FIG. 20B illustrates transfer curves of FIG. 20A over time represented as a 2D current heatmap.

FIG. 21A illustrates MSE and R² maps for the validation dataset from models with different V_{G} subsets.

FIG. 21B illustrates optimal V_{G} subset selected as a local extremum on MSE and R² maps.

FIG. 21C illustrates predictions on the validation dataset for the model with the optimal V_{G} subset.

FIG. 21D illustrates MSE and R² maps for the validation dataset from models with different time subsets.

FIG. 21E illustrates optimal time subset within V_{G} subset selected as local extremum on MSE and R² maps.

FIG. 21F illustrates model predictions on the validation dataset for the model with optimal V_{G} and time subsets.

FIG. 21G illustrates final model predictions on the blind testing dataset composed of 30 clinical samples from 3 different testing batches.

FIG. 21H illustrates R² values expanded over 3 testing batches for models with different input subsets.

FIG. 21I illustrates CV values for the optimal model expanded over different cholesterol ranges for blind tests.

FIG. 22A illustrates blind testing predictions for the model with the kinetic data from the entire operation range.

FIG. 22B illustrates blind testing predictions for the model with the kinetic data from the 2× optimal range.

FIG. 22C illustrates blind testing predictions for the model with the raw data from the optimal range.

FIG. 22D illustrates a table summarizing the performance of the different deep learning models. R² and CV summarized for deep learning models with different input data.

FIGS. 23A and 23B illustrates graphs of threshold voltage (Vₜₕ) as a function of time horseradish peroxidase (HRP) exposed to hydroperoxide (H₂O₂) (FIG. 23A) and TMB (FIG. 23B). In the presence of HRP, hydroperoxide (H₂O₂) oxidizes TMB to the colored product. Ab-HRP was not immobilized on the ITO surface but was free-floating in the solution with consistent concentrations of H₂O₂ and TMB over all solutions. Initial Vₜₕ in PBS without H₂O₂ and HRP was measured to be ca. 1.417 V. Vₜₕ slightly decreased to ca. 1.4 V for PBS and H₂O₂ mixture (still without Ab-HRP) due to acidic properties of H₂O₂. Vₜₕ began to decrease when 80 ng/mL Ab-HRP was added to PBS and H₂O₂ mixture as Ab-HRP produced protons from H₂O₂. The results in FIGS. 23A-23B also suggested that the detection system can translate Ab-HRP and TMB interactions. Pure TMB without Ab-HRP was initially acidic (pH of 4-5) which was estimated to be Vₜₕ of 1.2 V. No significant signal was observed from 16 ng/mL Ab-HRP in TMB, but Vₜₕ decreased with increasing Ab-HRP concentrations from 80 ng/ml to 10 g/mL.

### DETAILED DESCRIPTION OF ILLUSTRATED EMBODIMENTS

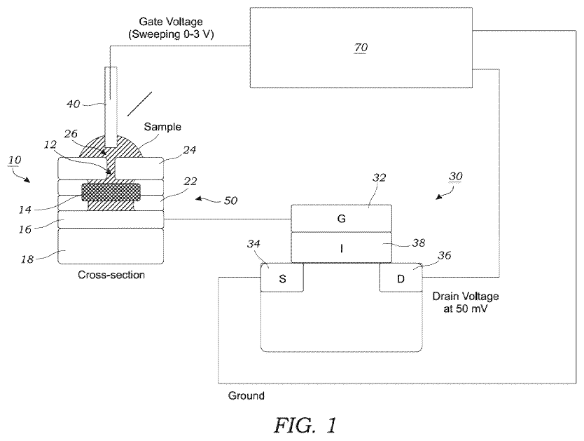

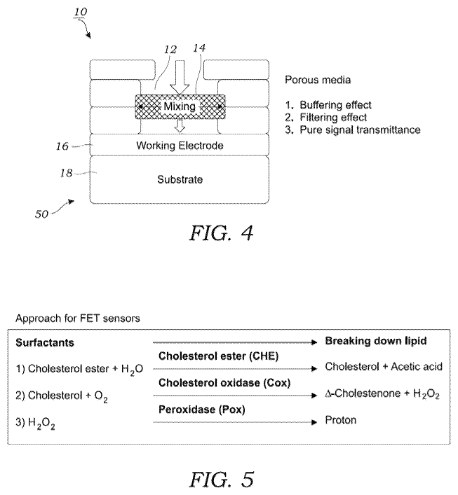

FIG. 1 illustrates a biosensor device 10 according to one embodiment. The biosensor device 10 is a single-use device that is used to analyze a fluid sample that is loaded into the same as explained herein. The biosensor device 10 includes a chamber 12 that receives a fluid sample. The chamber 12 may accommodate various volumes of fluids from microliter volumes to several milliliters. At least one porous sensing membrane 14 is disposed in the chamber 12. The porous membrane 14 extends across the chamber 12 so that any fluid sample that is loaded into the chamber 12 encounters and wets the at least one porous sensing membrane 14. The porous sensing membrane 14 may include, in some embodiments, a membrane. The porous sensing membrane 14 includes pores formed therein. The effective diameter or size of the pores may vary but typically falls within the range between 20 nm and 20 μm. The average pore size is more typically within the range of 100 nm to 20 μm. In some embodiments, the porous sensing membrane 14 has pore sizes that are substantially the same throughout the entire porous sensing membrane 14. In other embodiments, the porous sensing membrane 14 may have larger sized pores on one side of the porous sensing membrane 14 and smaller sized pores on the opposing side of the porous sensing membrane 14 (e.g., asymmetric structure or Janus membrane). The porous sensing membrane 14 extends across the width of the chamber 12 opening so that the fluid sample deposited into the chamber 12 comes into contact with an upper surface of the porous sensing membrane 14 and enters the pores contained therein. The porous sensing membrane 14 also contains therein or thereon one or more dried enzymes, surfactants, substrates, stabilizers, buffers, or reagents. These dried constituents may be applied using known methods of depositing dried reagents on a substrate. For immunoassay applications, the porous sensing membrane 14 may contain enzyme-labeled antibodies. The porous sensing membrane 14 may be paper-based, polysulfone (PSF), polyestersulfone (PES), regenerated cellulose, nitrocellulose, or the like. Mixing and filtration may take place in the porous sensing membrane 14 when exposed to the fluid sample as illustrated in FIG. 4.

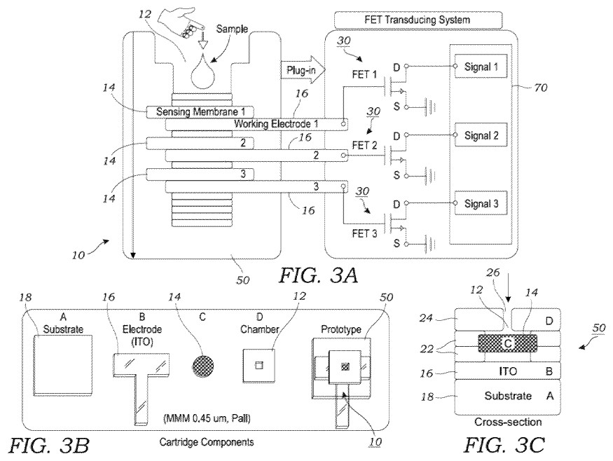

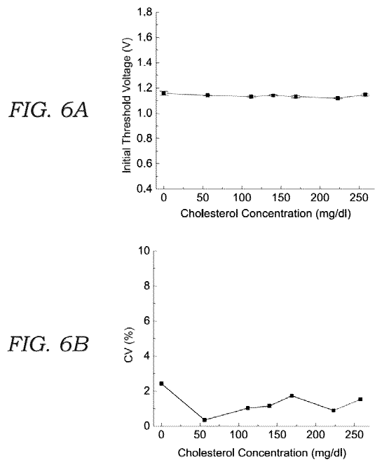

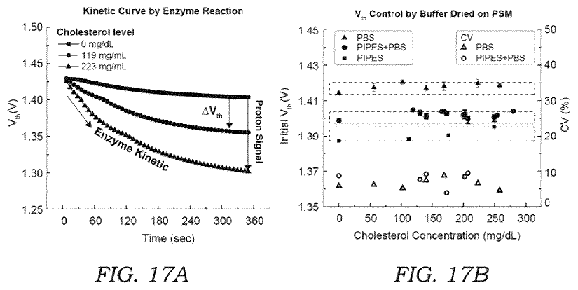

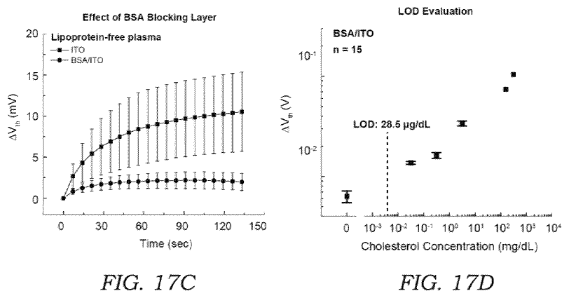

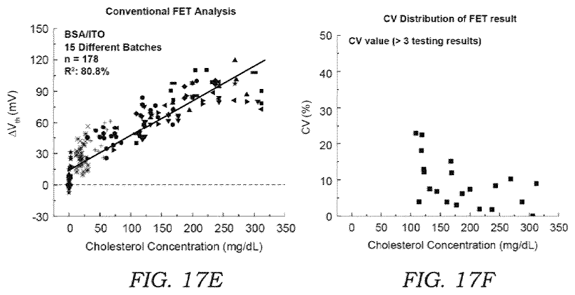

The porous sensing membrane 14 is disposed above and spaced apart from a working electrode 16 as best seen in FIGS. 1, 3C, and 4. The physical and chemical filtering that takes place in the porous sensing membrane 14 removes biofouling effects on the working electrode 16 and prevents larger molecules from interfering with the signals generated by the working electrode 16. The space or gap that is formed between the porous sensing membrane 14 and the working electrode 16 is used to accumulate protons or charged molecules (e.g., ions) that are transmitted to the working electrode 16 which increases the reproducibility, reliability, and stability of the biosensor device 10 free from sample matrix effects (see e.g., FIGS. 6A-6B and 17A-17F). In a typical arrangement, the biosensor device 10 is used in a vertical configuration where the opening to the chamber 12 is at the top of the device. The working electrode 16 is spaced apart from a lower surface of the porous sensing membrane 14. The space or gap may be several micrometers to tens of micrometers. A typical space or gap may be within the range of 10 μm to 100 μm and, in other embodiments, within the range of 10 μm to 50 μm. In one embodiment, the working electrode 16 is held atop a base 18 of the biosensor device 10. The base 18 may include a substrate layer on which other layers of the device are stacked. The size of the working electrode 16 may vary and may not cover the entire cross-sectional area of the chamber 12. This is particularly so in the multiplex embodiments discussed herein (FIG. 3A) where fluid continues to flow past a first working electrode 16 and then encounters further porous sensing membrane 14 and working electrodes 16. The working electrode 16 may be made from indium tin oxide (ITO), tin oxide, a metal oxide semiconductor, graphene, reduced graphene oxide, zinc oxide, conductive polymers such as regioregular poly(3-hexylthiophene-2,5-diyl) (P3HT), poly(3,4-ethylenedioxythiophene) polystyrene sulfonate (PEDOT:PSS) or other conductive 2D nanomaterials such as molybdenum disulfide. The entire biosensor device 10 may be in the form of a cartridge 50 or the like as illustrated in FIGS. 1, 2A, 2B, 3A-3C. The cartridge 50 may formed from a stacked or laminate configuration. For example, the cartridge 50 (FIGS. 1, 3C, 4) may have a bottom base 18 that holds the working electrode 16 in a spaced apart configuration away from the porous sensing membrane 14. Spacer layers 22 which may include tape layers in some embodiments may hold the porous sensing membrane 14 within the chamber 12 and define the gap or space between the lower surface of the porous sensing membrane 14 and the working electrode 16. A top layer 24 with an inlet 26 (e.g., opening) may be used as the top of the cartridge 50 and provides access to the chamber 12 for the fluid and access for the reference electrode 40 as explained herein.

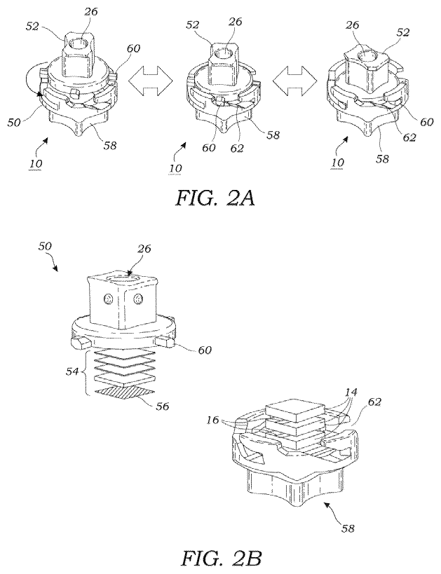

The cartridge 50 may, in some embodiments, be in a two-part cartridge like that illustrated in FIGS. 2A and 2B which are the brought together (e.g., twisted) to form the fully assembled biosensor device 10. For example, an upper half 52 of the cartridge 50 may contain a receptacle that receives the fluid sample. The upper half of the cartridge 50 may also include one or more filters, absorbing pads, diffusion layers 54 to pre-process fluid as seen in FIG. 2B. These may be supported on a supporting member 56 as seen in FIG. 2B. The lower half 58 of the cartridge 50 may contain the one or more porous sensing membrane 14 and the one or more associated working electrodes 16. In this embodiment, the upper half 52 of the cartridge 50 is twisted into the lower half 58 of the cartridge 50 using posts, detents, or bosses 60 located on the upper half 52 of the cartridge 50 that interface with respective recesses 62 in the lower half of the cartridge 50 to form the fully assembled biosensor device 10. Of course, other attachment scheme between the two-part cartridge 50 may be used including, for example, snaps, clips, press-fit, fasteners, and the like.

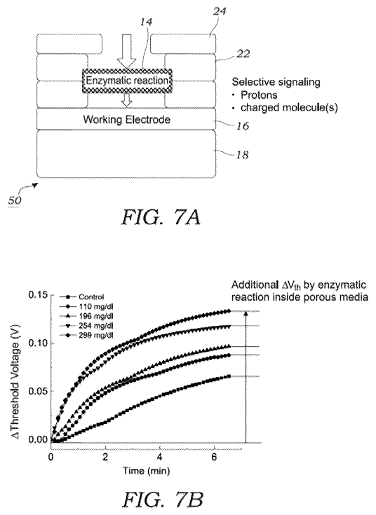

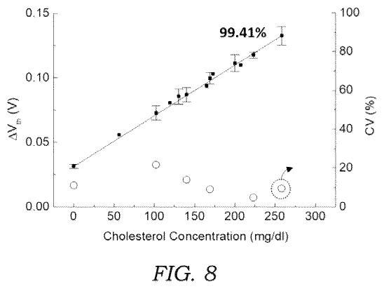

The working electrode 16 is electrically coupled to a FET 30 as seen in FIG. 1. The FET 30 includes a gate electrode 32, source electrode 34, drain electrode 36, and an insulator layer 38. Specifically, the working electrode 16 is electrically coupled to the gate electrode 32 of the FET 30. The protons or other charged species that are generated in the space or gap are then measured with the working electrode 16 that is coupled to the FET 30 (FIG. 7A). The FET 30 is, in one embodiment, contained in a FET readout system or other circuitry that is separate from the biosensor device 10. In this regard, the “wet” chemistry takes place in the chamber 12 of the biosensor device 10 while the FET operation takes place in a “dry” state. The working electrode 16 may be connected to the gate electrode 32 using a wire or other conductor.

As seen in FIG. 1, the FET 30 is electrically connected to a semiconductor parameter analyzer or current-voltage circuitry 70 that functions as the FET readout system and is used to measure the voltage and/or current response of the FET 30 which is used to detect and/or quantify a biomarker or analyte/target contained in the fluid sample that is loaded into the biosensor device 10. The biomarker or analyte/target interacts with the species contained in the porous sensing membrane 14 and generates charged species (e.g., H+ ions or other charged species, ions, molecules, or chemical substrates) that accumulate in the space or gap between the porous sensing membrane 14 and the working electrode 16 and measured using the FET-coupled working electrode 16. A reference electrode 40 that is configured to contact the fluid in the chamber 12 is also coupled to the semiconductor parameter analyzer or current-voltage circuitry 70 and is used to measure the voltage and/or current response of the FET 30. FIG. 1 illustrates the semiconductor parameter analyzer or current-voltage circuitry 70 coupled to a FET 30. The reference electrode 40 may include an Ag/AgCl reference electrode, graphite (or carbon), and platinum and is used to apply the gate voltage. As seen in FIG. 1, in one embodiment, the gate voltage is swept between 0 and 3V and the drain voltage is maintained at 50 mV.

In one embodiment, as explained herein, a change in the threshold voltage (Vₜₕ) is used to quantify the amount of the biomarker or analyte in the fluid sample. Vₜₕ is the minimum voltage that is required to essentially turn on the FET 30 and allow current to flow between the drain electrode 36 and source electrode 34 of the FET 30. In one embodiment, the change in the Vₜₕ at a particular time after introduction of the fluid sample into the biosensor device 10 is used to output the concentration of the biomarker or analyte. This may be several minutes after sample introduction. The change in Vₜₕ is measured as the change in Vₜₕ from the introduction of the fluid sample to a specified amount of time. Typically, the change in Vₜₕ is measured within about 7 minutes of introduction of the fluid sample.

The sample fluid that may be tested with the biosensor device 10 may include a body fluid such as blood, blood plasma, or blood sera. Other body fluids include saliva, sweat, urine, tears, and the like. In one particular embodiment, blood from a finger-prick may be loaded into the chamber 12 of the biosensor device 10. This allows the biosensor device 10 to be used in home settings. In some embodiments, the chamber 12 may include one or more additional media prior to (i.e., above) the porous sensing membrane 14. For example, the additional media may be used to filter cells or other particles in the sample (e.g., red blood cells) prior to entering the porous sensing membrane 14.

FIGS. 2A-2C illustrates an alternative embodiment for multiplex testing of multiple biomarkers or analytes in a biosensor device 10. In this embodiment, the chamber 12 includes a plurality of porous sensing membranes 14 stacked vertically within the chamber 12. Each porous sensing membrane 14 is associated with a respective working electrode 16. Thus, there are pairs of porous sensing membranes 14 and associated working electrodes 16 disposed in the chamber 12 and oriented vertically. A gap or space is present between the each of the porous sensing membrane 14 and the respective working electrodes 16. Sample fluid is loaded into the top of the chamber 12 and fluid progresses downward in the direction of the arrow whereby separate reactions take place within the respective porous sensing membrane 14 and the resulting ions or charged species are detected with the associated working electrode 16. Each working electrode 16 is electrically coupled to separate FETs 30 in the FET readout system and generates signals (e.g., voltage and/or current signals as explained herein) which are used to detect the concentration of the particular biomarkers or analytes. Wires or other conductor may be used to couple the working electrodes 16 to respective gate electrodes 32 of the different FETs 30. A reference electrode 40 is also configured to contact the fluid in the biosensor device 10 as in the prior embodiment. In some embodiments, a two-part cartridge like that illustrated in FIG. 2A may be used to form the fully assembled biosensor device 10.

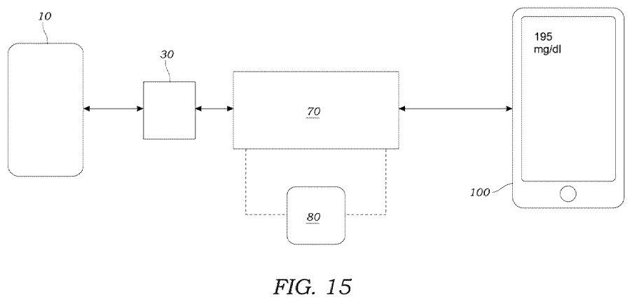

With reference to FIG. 14, to use the biosensor device 10, as seen in operation 200, a fluid sample is loaded into the chamber 12 of the biosensor device 10 via the inlet 26. The fluid sample may optionally be filtered or pre-processed prior to exposure to the one or more porous sensing membrane 14 as discussed herein. For example, cells or solid matter may be filtered out prior to fluid passing downward to the one or more porous sensing membrane 14. The fluid sample which contains the biomarkers or analytes then contacts the one or more porous sensing membrane 14 located in the chamber 12. Enzymatic or other reactions take place within the one or more porous sensing membrane 14 using the dried constituents contained therein or thereon. The electrically charged species (e.g., protons, ions, charged substrate(s), or charged molecules) are then accumulated in the respective space(s) or gap(s) located adjacent to the one or more working electrodes 16 associated with the one or more porous sensing membrane 14. The electrical response of the FETs 30 in the FET readout system coupled to the one or more working electrodes 16 is then analyzed. This may include monitoring, for example, the change in Vₜₕ or over time as seen in operation 210 of FIG. 14. This change in Vₜₕ (or equivalent parameter such as E_{ITO} which is discussed below) is then used to quantify the amount of the biomarker or analyte in the fluid sample. This may include looking at the kinetical curves or evolution of Vₜₕ over time or this can be looking at the particular change in Vₜₕ at a particular time point after loading the sample (see e.g., FIG. 7B). Generally, results should be available to the user within several minutes. As an alternative to change in Vₜₕ, one may look at the change in initial drain current at constant voltage for the FETs 30. As seen in operation 230, a concentration of the biomarker(s) or analyte(s) is then output or generated for the user. This may be displayed, for example, a computing device 100 such as a Smartphone as illustrated in FIG. 15. The computing device 100 may communicate with the semiconductor parameter analyzer or current-voltage circuitry 70 using a wired or wireless connection (e.g., Bluetooth, Wi-Fi, etc.). In some embodiments, the change in Vₜₕ may be used directly generate concentration value. For example, empirically derived data may be stored on software/memory of the computing device 100 or the semiconductor parameter analyzer or current-voltage circuitry 70 and can be used to generate the concentration value. This may be stored in form of a look-up table or function that directly calculates or generates the biomarker or analyte concentration from the Vₜₕ value.

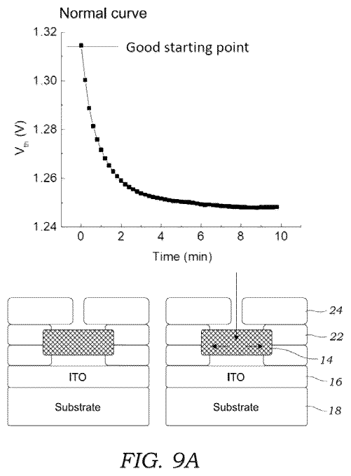

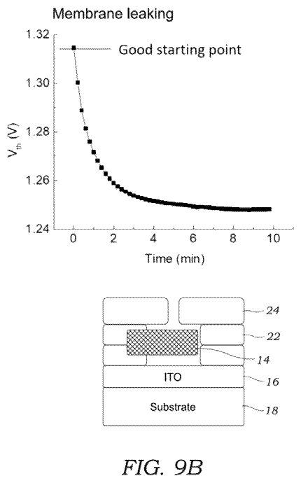

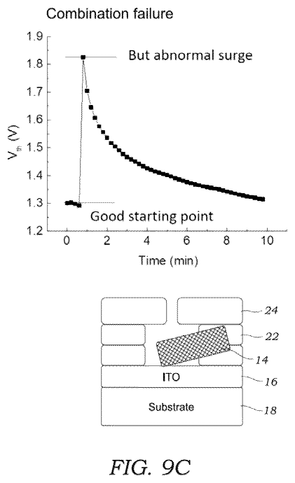

In some embodiments, the respective signals or data generated from the FETs 30 may be input to a trained neural network 80 as seen in FIG. 14 (operation 220) and FIG. 15 to provide more accurate or quicker concentration readings for the biomarkers or analytes. The trained neural network 80 may also be used for quality control purposes to check for test errors. For example, with reference to FIGS. 9A-9C, leaking of the porous sensing membrane 14 may be detected looking for sharp or rapid changes in Vₜₕ. Other failures or problems with testing may be identified by examining the evolution of the FET response.

In one embodiment, the trained neural network 80 uses kinetic data for quantitative biosensing of biomarkers or analytes. The challenges faced by existing FET biosensors, such as sample matrix interference in plasma and batch variations of cartridges, are effectively addressed by deep learning (DL) analysis, utilizing a comprehensive analysis of kinetic data extracted from target-specific bioreactions characterized through FET transfer curves obtained from the FETs 30. A proof-of-concept demonstration established that systematic DL-based analysis showcases a coefficient of variation <6.46% and an accuracy (r²>97.6% blindly compared to results obtained from a CLIA-certified clinical laboratory).

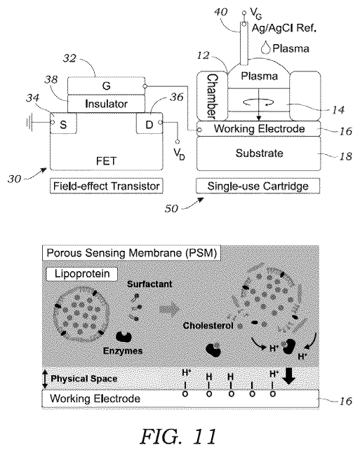

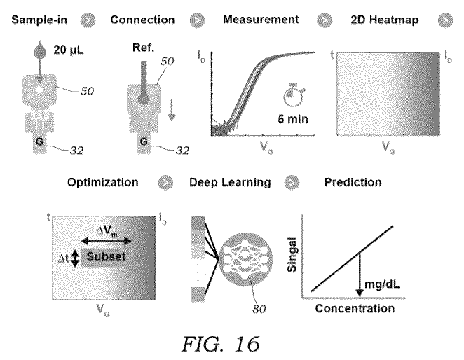

To evaluate DL performance with a trained neural network 80, a biosensor device 10 was constructed that designed to measure cholesterol, a standard biomarker in blood testing at clinics. The porous sensing membrane 14 was desiccated with enzymes such as cholesterol esterase (COE), cholesterol oxidase (COx), and peroxidase (POx), along with surfactants, stabilizers, and buffers, eliminating the need for additional functionalization steps. FIG. 11 schematically illustrates the setup for the cholesterol measuring biosensor 10. The working electrode 16, such as indium-tin-oxide (ITO), was positioned beneath the porous sensing membrane 14 within the cartridge 50, with ~50 μm of physical space or gap between the working electrode 16 and the porous sensing membrane 14. Injecting 20 μL of plasma into the cartridge's inlet 26 established a connection between the ITO working electrode 16 and the FET gate 32. Once the plasma contacted the porous sensing membrane 14, surfactants broke down lipoproteins, and a series of enzymatic reactions produced protons released into the physical spaces between the porous sensing membrane 14 and ITO working electrode 16. The real-time release of protons resulting from a series of enzymatic reactions specific to the cholesterol concentration in the plasma was continuously recorded in FET transfer curves repeatedly measured over 5 minutes with a semiconductor parameter analyzer or current-voltage circuitry 70 (FIG. 16).

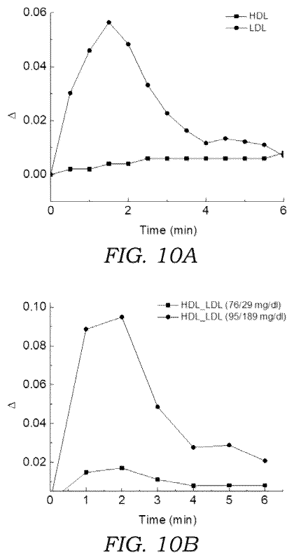

A variety of biomarkers or analytes may be analyzed using the biosensor device 10. Examples include low-density lipoprotein cholesterol (LDL-C) and/or high-density lipoprotein cholesterol (HDL-C) (FIGS. 10A-10B). Additional examples include, by way of illustration and no limitation, thyroid-stimulating hormone (TSH), glucose, triglyceride, creatinine, albumin, blood urea nitrogen, glycated hemoglobin, sodium, potassium, calcium, chlorine, phosphate, carbon dioxide, Alanine transaminase (ALT), Aspartate transaminase (AST), Alkaline phosphatase (ALP), bilirubin, Gamma-glutamyltransferase (GGT), progesterone, estrogen, estradiol, luteinizing hormone, or Anti-Mullerian hormone (AMH), COVID-19, influenza, HIV biomarkers, cancer biomarkers, disease biomarkers, or other pathogens.

### EXPERIMENTAL

### Results and Discussion

Operation Principle. The system combines a FET 30 with dry chemistry to achieve a new diagnostic platform with simple test logistics as seen, for example, in FIGS. 17A-17F which illustrates a cholesterol assay. The biosensor device 10 includes porous sensing membrane 14 (e.g., membrane) incorporates multiple assay components in a single-use cartridge 50, such as enzymes, surfactants, substrates, stabilizers, and buffers, all of which were dried in a void space of the porous sensing membrane 14 without any further procedures. After injection of ~15-20 μL plasma fluid into an inlet 26 of the cartridge 50, the FET 30 remotely measures electroactive enzymatic reactions on the porous sensing membrane 14 in the cartridge 50. An ion-sensitive working electrode 16, which in this embodiment was indium-tin-oxide (ITO), is built underneath the porous sensing membrane 14 in the cartridge 50 and receives protons in real-time as a target signal remotely delivered to a gate electrode 32 of the FET 30 via an electrical connection between working electrode 16 and the gate electrode 32 of FET 30. The FET 30 quantifies cholesterol via changes in the Vₜₕ of the FET 30. In one embodiment that uses a trained neural network 80 to improve results, the transfer curves acquired with the semiconductor parameter analyzer or current-voltage circuitry 70 were transformed into a 2D heatmap, encapsulating all enzymatic kinetic details characterized in the sum of transfer curves. The DL analysis further optimized the subset of kinetic signals carrying concentration-specific data to quantify cholesterol concentrations in patient plasma samples as seen in FIG. 16.

Once the plasma under test is introduced in the porous sensing membrane 14, all dried assay components dissolve in the sample, and surfactants begin to break down lipoproteins. Following a series of enzymatic reactions (FIG. 5) from cholesterol esterase (CHO), cholesterol oxidase (COx), and peroxidase (POx), protons are generated as a final product (FIG. 11). The protons are collected in a physical gap (~58 μm) held between the porous sensing membrane 14 and the working electrode 16 formed by a double-sided tape which is used as a spacer layer 22. Primarily, the porous sensing membrane 14 serves as a physical filter for diverse types of remaining proteins in plasma through small-sized pores (~20 nm) of the porous sensing membrane 14, which prevents non-specific binding signals on working electrode 16. Second, strong buffers and stabilizers already dried on the porous sensing membrane 14 control the pH of plasma regardless of the sample matrix and stabilize the system for optimal enzyme reactions. From two main effects above, the purified enzymatic signals are delivered to the space or gap between the porous sensing membrane 14 and the working electrode 16 for specific signaling.

pH sensitivity of ITO. Protons are the target signal for the device 10 in response to cholesterol in plasma. Thus, pH sensitivity of ITO was initially evaluated as a baseline. In classic semiconductor physics, Vₜₕ of the remote-gate FET (RGFET) is a sum of the electric potentials of each component in the RGFET:

$${V}_{th} = {V}_{FET} + {E}_{ref} + {ϕ}_{lj} - {φ}_{s} + {χ}_{sol} + \frac{{ϕ}_{RG}}{q} \qquad ( 1 )$$

where V_{FET} is Vₜₕ of a FET without the RG, E_{ref} is the absolute potential of the reference electrode, φₗⱼ is the liquid junction potential difference, φₛ is the surface potential at the electrolyte/sensing film interface, χₛₒₗ is the electrolyte insulator surface dipole potential, and φ_{RG} is the work function (in energy units) of the RG material. V_{FET} (1.51 V) and E_{ref} (0.316 V) are consistent over all experiments. The standard electric potential of ITO (E_{ITO}) was defined within equation (1) which is only associated with properties of intrinsic electric potentials of the overlying solution, RG material and its contact.

$${E}_{ITO} = {ϕ}_{lj} - {φ}_{s} + {χ}_{sol} + \frac{{ϕ}_{ITO}}{q} \qquad ( 2 )$$

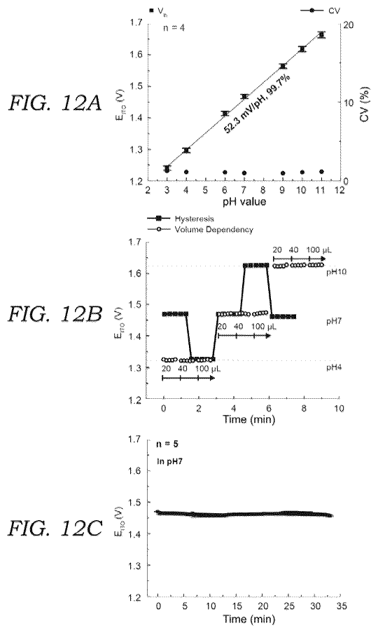

Therefore, E_{ITO} is an intrinsic electric potential of ITO in response to pH of the solution on ITO. FIG. 12A shows E_{ITO} distribution measured for each pH over four different ITO surfaces. pH sensitivity calculated as the slope of pH versus E_{ITO} was evaluated to be 52.3 mV/pH with an R² of 99.7%. A device-to-device variation was insignificant while all CV values of E_{ITO} for each pH is no more than 15%; the increasing trend in CV for higher pH was mainly attributed to smaller averaged values of E_{ITO} for CV calculation while standard deviation remains in a similar range. Hysteresis was tested by a rapid loop of pH 7-4-7-10-7, which showed a reversible tendency with changing pH values (FIG. 12B). The difference between the initial value of E_{ITO} at the first cycle of pH7 and the final E_{ITO} at the final cycle of pH 7 was 8 mV (FIG. 12B). Due to a significantly higher input impedance of the FET 30 than that of an ITO RG module, there are no changes in E_{ITO} with increasing contact areas of media volume size on the ITO surface, but the system specifically shows clear responses to proton concentrations (FIG. 12B). Insignificant drift in FIG. 12C suggests that the ITO working electrode 16 is highly stable to translate enzymatic reactions specifically; the drift rate was measured to be 8 μV/min over 5 different ITO SEs.

Cholesterol Detection. For cholesterol detection, the FET 30 included the porous sensing membrane 14 on the top of the working electrode 16 with a physical gap in FIG. 11. Enzymes, surfactants, substrates, stabilizers, and buffers were embedded in porous sensing membrane 14 with a simple fabrication step without needs of a complex surface chemistry. This involves drying a sensing solution (SS) over the porous sensing membrane 14. This design also enabled the porous sensing membrane 14 to transmit purified enzymatic signals to the working electrode 16 while minimizing sample matrix effects and increasing the specificity of target signals.

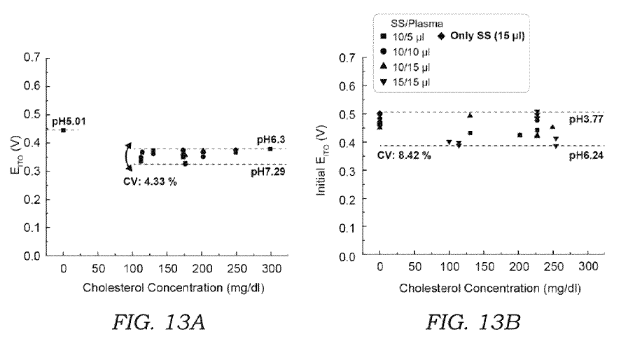

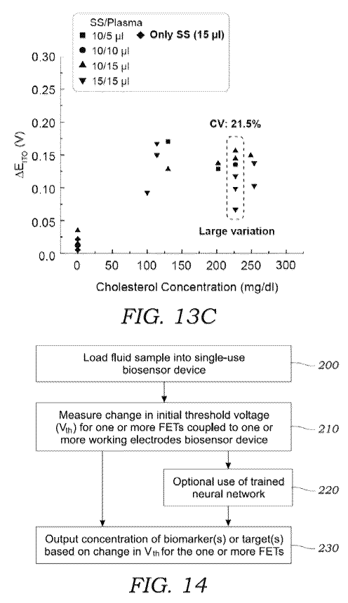

FIG. 17B shows the initial Vₜₕ distribution of cartridges 50 controlled by each buffer of PBS, piperazine-N,N′-bis(2-ethanesulfonic acid) (PIPES), and PIPES/PBS (5:5), dried on each porous sensing membrane 14; the rest of porous sensing membrane components remained the same. The initial Vₜₕ is Vₜₕ before starting enzymatic reactions, ideally. Specific buffer in porous sensing membrane 14 determined the levels of Vₜₕ for a certain range in FIG. 17B, regardless of concentrations of cholesterols and pH of plasma shown in FIG. 13A; pH of testing plasma was measured to be from 6.30 to 7.29 on bare ITO (FIG. 13A) based on pH curves in FIG. 12A. CV of initial E_{ITO} from all plasma samples but except for lipoprotein-deleted plasma was measured to be 4.33% with low variance (FIG. 13A). Lipoprotein-deleted plasma (cholesterol-free plasma) was purchased separately for controls, with a measured pH of 5.01. Interestingly, even lipoprotein-deleted plasma with much lower pH poses at specific levels of initial E_{ITO} controlled by specific buffer of the porous sensing membrane. This result supports that plasma had a chance to react within the porous sensing membrane 14 first and the resultant mixture was transmitted to the space between the porous sensing membrane 14 and the working electrode 16. The resultant mixture controlled by properties of porous sensing membrane 14 produced ΔVₜₕ from each initial Vₜₕ due to generation of protons.

In contrast to the biosensor device 10 described herein, a wet chemistry approach (FIG. 13B) is challenging to implement with clinical sample environments. FIG. 13B shows distribution of initial E_{ITO} of clinical plasma samples mixed with a sensing solution (SS) that is a mixture of enzymes (CHO, COx, POx) and surfactant (Triton X-100) dissolved in PBS/PIPES buffers. It is noted that all compositions and components of SS remained the same to cases of FIGS. 12A and 12B but solution phase without porous sensing membrane 14. SS was added on the top of plasma samples placed on the bare ITO electrode. It is noted that there was no clear dependency between volumes of SS or plasma and levels of initial E_{ITO} rather being randomized distributions (FIG. 13B). CV of all initial E_{ITO} values was measured to be 8.42%. Such a larger distribution in initial E_{ITO} than those in FIG. 13A stemmed from different mixing dynamics for a wet phase between SS and each plasma with different viscosities and ionic components. The different mixing rate enlarged pH variation of mixtures while sample matrix effects began to appear.

In FIG. 13C, ΔE_{ITO} distribution by wet chemistry approach is displayed; pure SS and SS with cholesterol-free plasma showed ΔE_{ITO} less than 35 mV. Compared with those controls, clinical plasma showed larger shifts in ΔE_{ITO}, suggesting that enzymatic reaction was at least reflected by ΔE_{ITO}. Linear signal trend in ΔE_{ITO} was vaguely seen for mixing conditions of SS/plasma (10/15 μL) but the rest of the groups were not. In contrast to FIG. 12B, ΔE_{ITO} is affected by the volume size of SS because different enzyme concentrations would result in different quantities of protons for the same plasma. For a conventional FET sensing platform, controlling exact concentrations of enzymes and volume size of plasma samples is extremely challenging without either precise microfluidic channels or surface chemistry to functionalize exact amounts of biological probes such as enzymes on the surface. CV of E_{ITO} from the specific plasma sample with 227 mg/dL cholesterol even increased up to 21.5% as the SS/plasma ratio varied.

FET Data. Upon injecting human plasma into the cartridge 50, distinct real-time signal patterns were observed in response to varying cholesterol levels, as indicated by changes in the Vₜₕ (ΔVₜₕ) relative to the Vₜₕ of lipoprotein-free cholesterol plasma (FIG. 17A). The protons generated by each electroactive enzymatic reaction decreased Vₜₕ levels of n-type FETs 30 due to the positive surface potentials applied on the ITO working electrode 16 from protons. The initial Vₜₕ of the cartridges 50 was largely influenced by the pH and ion concentration of human plasma, as well as batch variations in cartridges 50, along with diverse proteins and components in plasma that could cause non-specific binding on the ITO working electrode 16 surface. Despite injections of different plasma samples, the initial Vₜₕ values tended to overlap (FIG. 17A). This could be attributed to the cartridge design, which incorporated a ~50 μm air gap between the porous sensing membrane 14 and the ITO working electrode 16 (inset of FIG. 17A). The presence of this air gap played a pivotal role in facilitating the mixing process between the porous sensing membrane 14 and plasma samples. It ensured that the mixing process occurred before the original plasma came into direct contact with the ITO working electrode 16. As a result, any sample matrix effects were significantly diluted by the potent buffer components that were already desiccated within the porous sensing membrane 14. Consequently, the porous sensing membrane 14 efficiently transmitted purified electroenzymatic signals to the ITO working electrode 16 surface.

FIG. 17B further supports the fluidic dynamics in the cartridge 50 described above by demonstrating the controlled initial Vₜₕ of each cartridge 50 after the injection of different random human plasma samples using various buffer solutions, such as phosphate-buffered saline (PBS), piperazine-N,N′-bis(2-ethanesulfonic acid) (PIPES), and PIPES/PBS (5:5 ratio), dried onto identical PSMs, with the remaining PSM components being consistent. Small variations in the initial Vₜₕ levels are shown for each group of cartridges dried with PBS, PIPES/PBS, and PIPES (CV<10%). Even lipoprotein-free plasma samples with a pH of 5 tend to be controlled by the buffer solution in the PSM of the cartridge (FIG. 17B). For subsequent experiments, PBS (pH 7.4) was selected as the buffer component for the porous sensing membrane 14 to minimize the pH disparity between plasma (pH 7.35 to 7.45) and the dried buffers.

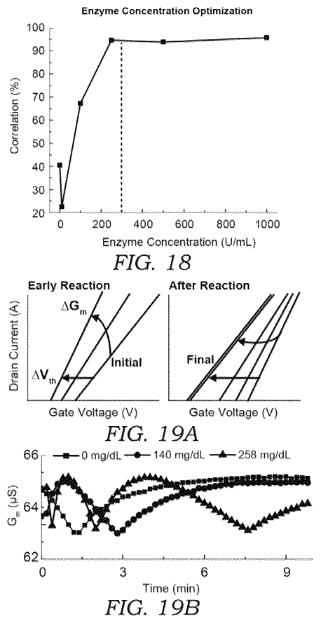

The concentrations of COE, COx, and POx for the porous sensing membrane 14 were optimized by evaluating correlations between the enzyme concentrations and the cholesterol signals (FIG. 18). Concentrations of enzymes exceeding 200 U/mL displayed saturated correlations, resulting in 300 U/mL enzyme concentrations for COE, COx, and POx being chosen in all subsequent experiments. However, without the POx enzyme, no correlative sensing signals were obtained, implying that POx played a critical role in producing electroactive enzymatic signals such as protons. It is noted that the enzyme solutions lacked long-term shelf life without drying them on the porous sensing membrane 14, as shown by a large drift in the initial Vₜₕ levels of the enzyme solution over time.

It was further discovered that the use of bovine serum albumin (BSA) coating on the ITO working electrode 16 significantly mitigated sample matrix effects without compromising the detection signals (FIG. 17C). In contrast, bare ITO working electrodes 16 without BSA coating exhibited larger shifts in electroenzymatic signals, even for free cholesterol plasma samples. This might be due to the remaining proteins and ions in lipoprotein-free plasma causing non-specific signals by interacting with the bare ITO surface.

The LOD achieved by the biosensor device 10 was assessed in FIG. 17D using diluted clinical plasma with lipoprotein-free plasma, ensuring controlled conditions. The estimated LOD for cholesterol is determined to be 28.5 μg/dL (737 nM). This LOD range is previously exclusively demonstrated by conventional electrochemical detection methods employing sophisticated and complex device fabrications, including the utilization of nanomaterials and mediators. The highly sensitive attribute of FET sensors extends this remarkable LOD range to paper-based analytical devices, highlighting the efficacy of the platform even without the need for functionalization.

Lastly, in FIG. 17E, the distribution of ΔVₜₕ values is shown for cholesterol concentrations measured from patient plasma samples across 178 cartridges 50 produced during 15 different sub-batch fabrications. Notably, when enzymes were absent from the porous sensing membrane 14, no correlated detection signals were observed, leaving sample matrix effects the only reason for minor variations in ΔVₜₕ. While there was a degree of correlation between cholesterol levels and ΔVₜₕ values, relying solely on ΔVₜₕ (which is conventionally used in FET sensors analysis to determine target-signals) as the sensor readout presented major challenges. When applied to the experimental dataset, this standard approach displayed a low R² of 80.8% (FIG. 17E) and a large CV of up to 22.8% within a clinically relevant range (i.e. 100-150 mg/dL, FIG. 17F), confirming the challenges of FET biosensor performance on physiological samples, despite optimizations of the diverse fabrication factors listed above.

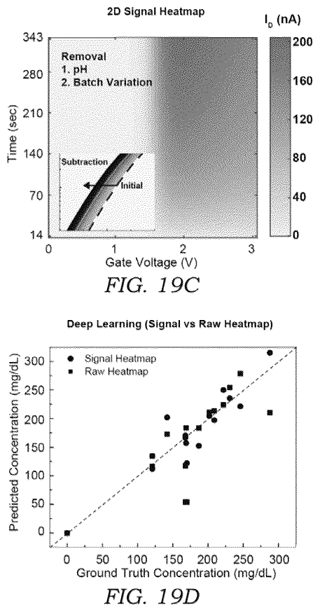

The conventional FET analysis, focusing solely on ΔVₜₕ (FIG. 17E) by considering the endpoint and initial point, may fall short of providing a comprehensive understanding of variations during enzyme reactions influenced by the time-dependent enzyme reaction rate (FIG. 19A) and sample matrix effects. Moreover, the real-time FET measurement (FIG. 17A), capturing a cumulative representation of specific snapshots at particular moments over time, also offers limited information about enzyme reactions. For instance, the Gₘ values, which cannot be obtained from real-time measurement approach in FIG. 17A, exhibit significant variations during reactions (FIG. 19B), These variations are influenced by factors such as the rate of enzyme reactions, the mixing process within the cartridge 50, and the presence of sample matrix effects. Interpreting these dynamic behaviors for each specific case can be challenging, underscoring the need for more advanced data-driven analytical techniques, such as DL, to comprehensively capture and interpret the dynamic nature of these biochemical processes, as detailed in the subsequent sections.

Design of DL-based Signal Analysis. DL benefits from the universal function approximation power of a trained neural network 80 to harness the complex non-linear kinetic data from the FET 30 sensor to measure the analyte concentrations. Here, DL-based analysis and trained neural networks 80 were employed for two key objectives: 1) optimizing the subset of kinetic signals carrying concentration-specific information, and 2) quantifying analyte concentrations in patient plasma samples. The DL models were structured as fully-connected shallow networks with three hidden layers, utilizing continuously measured FET transfer curves as the input data to the trained neural networks 80. Both the kinetic data input and the network architecture underwent optimization through a 4-fold cross-validation on the validation set of plasma samples. The optimized trained neural network 80 was subsequently blind-tested on 30 additional samples from the testing set, never used before.

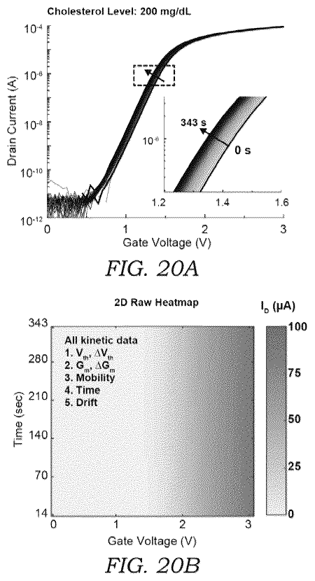

For DL analysis, transfer curves obtained with a semiconductor parameter analyzer or current-voltage circuitry 70 measured over 5 minutes for each plasma sample were transformed into a 2D heatmap (FIGS. 20A and 20B). This heatmap visually represented all enzymatic kinetic details, encompassing characteristics observed in the sum of raw transfer curves (FIG. 20A), such as drift during measurement, initial Vₜₕ, and changes in Vₜₕ, electronic mobility, and Gₘ due to enzyme reactions (FIG. 20B). Notably, preliminary observations underscored the significance of subtracting the initial transfer curve data from the raw heatmap in FIG. 20B. The initial transfer curves typically serve as a baseline for conventional FET analysis to measure the relative change in target-specific signals, which could be significantly affected by pH, ion-concentrations, and sample matrix effects. Thus, the raw heatmap (FIG. 19B), after subtracting the initial transfer curve properties and referred to as the signal heatmap 90 (FIG. 19C), encapsulated pure kinetic information primarily associated with enzymatic reactions, and remained, by and large, unaffected by interference from varying pH levels in plasma samples and batch variations. Using this signal heatmap 90 as input resulted in a substantial improvement in the neural network inference, reducing the CV from 20.1% to 8.5% and increasing the R² from 69.8% to 90.4% (FIG. 19D).

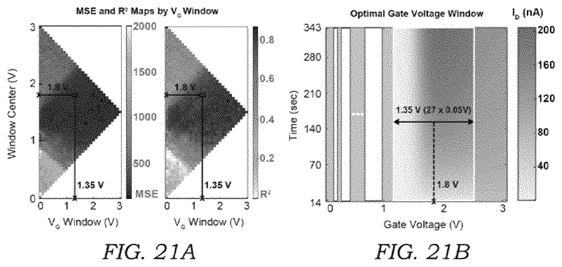

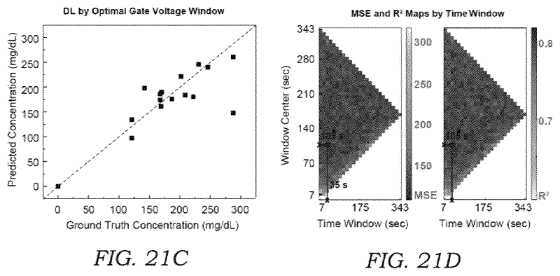

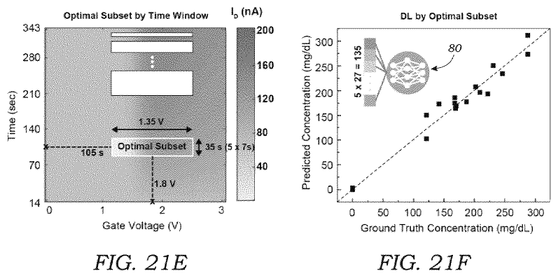

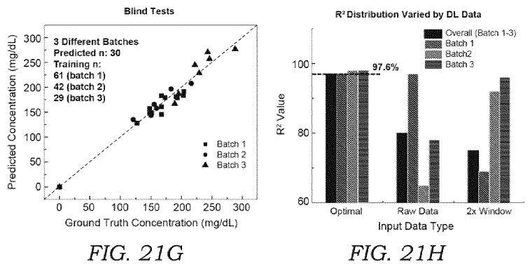

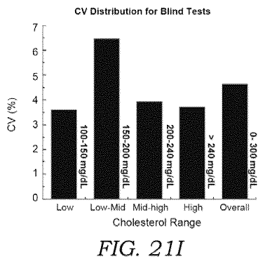

DL was further applied to optimize the subset of kinetic signals containing concentration-specific information within the signal heatmap 90. This optimization considered both the size of the V_{G} window (i.e., within the 0-3 V range, FIGS. 21A-21C) and the time window (i.e., within the 14-343 s range, FIGS. 21D-21F). The selection of the optimal model was based on achieving the lowest mean square error (MSE) and the highest R² values when comparing predicted and ground truth cholesterol concentrations for samples from the validation dataset, with variations in the sizes of V_{G} windows. Consequently, the optimal V_{G} window was identified to be between 1.15 V and 2.45 V (FIG. 21B). The predicted concentrations within this optimal V_{G} subset still exhibited a high CV of 20.7% and a low R² of 90.7%. With a fixed V_{G} window, an optimal time subset was determined to be within the range of 91-119 s (FIG. 21E). The trained neural network 80 utilizing the optimized subset in FIG. 21E demonstrated improved quantification performance on the same validation set in FIG. 21C, achieving an R² of 95.4% and a CV of 11.4% (FIG. 21F). The same trained neural network 10 was further utilized to generate blind testing results using plasma samples never seen before, which will be detailed in the next section.

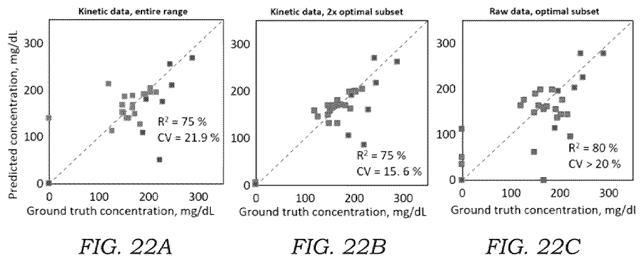

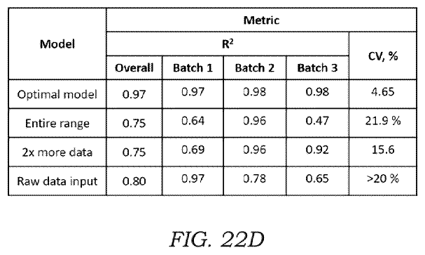

Blind Testing Results. The optimized neural network 80, incorporating the optimal architecture and refined kinetic data input, underwent blind testing to quantify cholesterol concentrations across 30 clinical plasma samples from three distinct testing batches (FIG. 21G). The blind testing predictions exhibited a high correlation with the ground truth cholesterol values, with R² exceeding 97.6% for all three batches (FIG. 21H). Additionally, the model demonstrated low variations in predictions, with a maximum CV of 6.46% over different cholesterol concentration ranges (FIG. 21I). Importantly, blind testing predictions from the neural network model 80 using the optimal subset in the signal heatmap 90 (FIG. 21G) outperformed other models, including the model using the entire raw heatmap (FIG. 22A), the model with a 2× larger window size than the optimal subset in the signal heatmap 80 (FIG. 22B), and the model using the optimal subset in the raw heatmap (FIG. 22C). Therefore, this method not only enhanced the understanding of enzymatic reaction kinetics but also improved the robustness of the network predictions, making them more resistant to variations induced by sample matrix effects.

It is noted that for each of the three testing batches, optimized neural network models were trained separately using samples from the same batch to minimize inter-batch variability. For the same blind testing set, a single model trained on samples from all three baches had inferior accuracy (R²: 88.6% excluding outliers) and precision (CV of 10.85%). Higher inference variations of this single neural network model between different batches originate from additional variabilities in porous sensing membrane 14 and reagent batches used during different testing days. In future iterations of assay development, batch-specific information along with the sensor data may be considered to create a more robust inference model that could generalize to different batches.

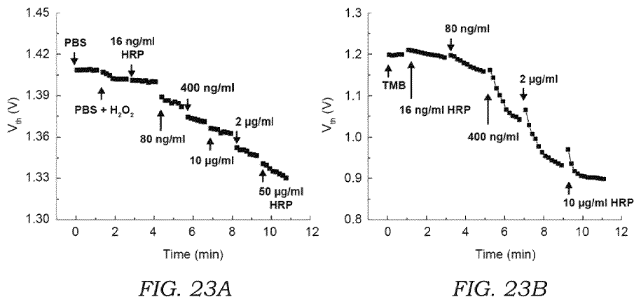

Scalability to Immunoassay. The biosensor platform has the potential to be adapted for immunoassays, which holds significant promise in a wide range of biomedical applications. To illustrate this adaptability, a proof-of-concept experiment was conducted involving electroactive enzymatic signaling on the biosensor 10 (FIGS. 23A-23B). In this experiment, the interaction between horseradish peroxidase (HRP)-labeled anti-mouse IgG (Ab-HRP), hydrogen peroxide (H₂O₂), and 3,3′,5,5′-tetramethylbenzidine (TMB) was used. This combination that has long been established and widely used in conventional enzyme-linked immunosorbent assays (ELISA) for detecting biomarkers in sandwich immunoassays using the resultant-colored product of TMB. TMB and HRP reactions also produce protons, which serve as a target signal in this detection platform. This phenomenon is depicted in FIGS. 23A-23B, where increased concentrations of Ab-HRP decrease Vₜₕ levels of the FET upon the injection of H₂O₂ (FIG. 23A) or TMB (FIG. 23B). TMB and HRP signaling can be integrated into a LFA framework, complemented by zones dedicated to capture antibody, detection antibody, and chemical substrate. This design is particularly beneficial for immunoassays that require high sensitivity, such as troponin I and metabolite assays, as well as for the rapid detection of infectious diseases.

The biosensor device 10 and systems disclosed herein offer a facile way to adopt dry chemistry for FETs 30 by integrating porous sensing membrane 14 on sensing zone of FETs 30. The approach eliminated conventionally demanding needs of FET biosensors such as surface chemistry and complex operation logistics without any complicated and expensive manufacturing processes. As a proof-of-concept, cholesterol detection was demonstrated in a range from 0 to 299 mg/dL in 20 μL plasma samples in a prototype cartridge 50, concurring with the results provided by FDA-approved clinical lab instruments (see FIG. 8). The limitation of a wet chemistry approach was identified by performing without functionalization of enzymes on the working electrode 16 such as non-linear signaling by matrix effects for physiological samples. This dry chemistry approach for FET-based sensors significantly advances the ability to commercialize a low-cost, miniaturized, and highly accurate blood testing devices without sample matrix effects. The biosensor device 10 may further deliver an at-home blood testing platform that uses of a single-use cartridge 50 and a palm-size computing device 100 (e.g., reader device) such as that seen in FIG. 15 for measuring comprehensive biomarkers of chronic diseases from a single droplet of blood such as hyperlipidemia, diabetes, and thyroid disorders, as part of diagnostic components of telehealth. It will significantly reduce the burden of medical/healthcare resource imposed by medical appointments and regular blood testing to follow the progress or management of each chronic disease.

### Experimental Procedures

Sensing Solution Preparation. A sensing solution included enzymes, a stabilizer, and buffer solutions. 300 U/mL cholesterol esterase (Toyobo, COE-311), 300 U/mL cholesterol oxidase (Toyobo, COO-321), and 300 U/mL peroxidase (Toyobo, POX-301) were dissolved in PBS, piperazine-N,N′-bis(2-ethanesulfonic acid) (PIPES), or PBS/PIPES (45/55% ratio) buffer solution, respectively. Triton X-100 (Sigma Aldrich, SLBM3869V), tween 20 (Surf's Up surfactant Kit, K40000), and 10% BSA (Thermo Scientific, WL335677) were mixed with the enzyme solution at a 0.5% (v/v) for each.

Cartridge Fabrication. ITO (Sigma Aldrich, 639303) cleaned with isopropanol for 20 min was utilized as the working electrode 16. ITO was further incubated with 10% BSA solution for 4 hours to achieve a blocking layer on the surface of the ITO working electrode 16. The final sensing solution described earlier was fully spread over each porous sensing membrane 14 made of an asymmetric super micron polysulfone membrane (Pall, T9EXPPA0045S00M) with a 0.45 μm average pore size and a nitrocellulose membrane with a 0.22 μm average pore size (Sartorius, 11327-41BL). Each porous sensing membrane 14 was fully dried for 20 min using nitrogen gas and stored under silica gel for 2 hours. Dried porous sensing membrane 14 was sliced to a 6 mm diameter circle for the cartridge component. The ITO working electrode 16 was taped on an acrylic sheet substrate (1 mm-thick, 1.5 by 1.5 cm) using double-sided tape. Another double-sided tape (50 μm thickness) was mounted on the ITO working electrode 16 with an opening window for porous sensing membrane 14 placement. The porous sensing membrane 14 and chamber 12 were sequentially added on the top of double-sided tape (FIGS. 3B-3C). The ITO working electrode 16 was connected to the gate 32 of MOSFET 30 using an alligator clip for measurements. All components, including ITO working electrode 16, acrylic sheet and double-side tapes, were fabricated by laser cutter (60 W Speedy 100 CO₂ laser, Trotec, USA).

Electrical Measurement System. The ITO working electrode 16 of the cartridge 50 was connected to the gate 32 of a commercial n-type metal-oxide-semiconductor field-effect transistor (MOSFET) 30 (CD4007UB) using an alligator clip. The same MOSFET 30 was used over all measurements consistently. A 20 μL volume of plasma was injected into the inlet 26 of the cartridge 50. An Ag/AgCl reference electrode 40 contacted the plasma, applying the V_{G} in a range from 0 to 3 V for all measurements. All transfer curves were measured using a Keithley 4200A semiconductor analyzer 70 with a source-drain voltage set at 50 mV, and the V_{G} fixed in the double-sweep mode. Transfer curves of the FET 30 were repeatedly measured for 5 min under each plasma sample. The Vₜₕ was calculated as the V_{G} corresponding to an ID of 1 μA in each transfer curve. Standard pH buffer solutions were used to evaluate the pH sensitivity of the ITO working electrode 16. Each solution was removed by pipetting after each measurement. For HRP response tests in FIGS. 22A, 22B, 10 mM H₂O₂ in PBS was mixed with IgG-HRP (Southern Biotech, 1030-05) on the bare ITO surface, sequentially, with increasing concentrations of IgG-HRP in a range from 16 ng/mL to 50 μg/mL in PBS. Also, TMB (Thermo Scientific, 34028) was added to goat IgG-HRP solution in a range from 16 ng/ml to 10 μg/mL in PBS before testing.

Clinical Sample Tests. Left-over lithium heparin plasma from patient samples collected at The University of Chicago Medical Center with cholesterol concentrations ranging from 100 to 300 mg/dL were de-identified and stored at −20° C. until use. Cholesterol concentrations were quantified using the Roche CHOL2 enzymatic colorimetric assay on the Roche Cobas 8000 analyzer system, c701 module (Indianapolis, IN, USA). After thawing, the samples were stored at 2-8° C. for up to seven days. Samples were collected under a quality assurance protocol, which qualified for an institutional review board waiver and no patient identifiers were utilized. Lipoprotein-free human plasma was purchased from Kalen Biomedical, LLC as control. In order to evaluate LOD (FIG. 17D), 312 mg/dL clinical plasma sample was diluted by lipoprotein-free human plasma. The CV values (FIG. 17E) were obtained from at least 3 testing cartridges 50 for the same human plasma sample.

A total of 179 plasma samples were tested within 3 testing batches, including 86 plasma samples in the first batch, 57 plasma samples in the second batch and 36 plasma samples in the third batch. In the first testing batch, 61 plasma samples were used for training, with 17 samples for validation and 8 samples for blind testing of the deep learning model. In the second batch, 42 samples were allocated for training, with 15 samples for blind testing, and in the third batch, 29 samples were reserved for training, with 7 samples for blind testing. This split was dictated by the uniform selection of training samples from different ranges of cholesterol concentration (in 100-300 mg/dL range).

Data Processing and Deep Learning Analysis. For each sample, the transfer curves of the FET 30 sensor were repeatedly measured over 49 cycles with 7 sec per cycle (i.e., a total of 343 s period). Before applying DL-based analysis, the first captured cycle was subtracted from all 49 cycles within the raw heatmap (FIG. 19B), yielding 48 cycles within the heatmap, termed signal heatmap (FIG. 19C). For DL analysis, the signal heatmap 90 was converted into a 1D array and input into the trained neural network 80. The neural network architecture was optimized through a 4-fold cross-validation on the validation set, and the optimal model represented a shallow neural network with a fully-connected architecture with 3 hidden layers (128, 64 and 32 units), each followed by batch normalization and 0.5 dropout. All three layers used ReLU activation functions and L2 regularization. The loss function (L) was MSE compiled with Adam optimizer, a learning rate of 10-3, and a batch size of 5, i.e.,

$$L = \frac{1}{N} {\sum }_{i = 1}^{N} {( {y}_{i} - {y}_{i}^{′} )}^{2} ,$$

where yᵢ are the ground truth analyte concentrations, y′ₗ are the predicted concentrations, and N is the batch size.

The input signals into the neural network 80 were further optimized by selecting a subset of current values from the total operating range (i.e., 14-343 s time range and 0-3 V V_{G} range). The optimization was done in two steps through a 4-fold cross-validation (see Deep learning-based optimization of the kinetic data for more details) on 17 samples from the validation set. The optimized model architecture for the ultimate trained neural network 80 (i.e. model with optimal input subset and architecture) was further used at the blind testing phase.

Blind testing set included 30 samples (i.e. not seen during network optimization) from three different testing batches. For each batch, the final optimized models were independently trained using samples from the same batch (see Clinical Sample Tests). Training times for batches 1 to 3 were 113 s, 143 s, and 145 s, respectively. Irrespective of the batch number, blind testing of the trained model averaged 110 ms per sample for a batch size of 1, and this time decreased to 35 ms per sample when using a batch size of 10. Data preprocessing and training/testing of neural networks were performed in Python, using OpenCV and TensorFlow libraries. Training/testing of the neural networks was done on a desktop computer with a GeForce GT 1080 Ti (NVIDIA).

Deep learning-based Optimization of the Kinetic Data. The neural network input optimization process was performed through a 4-fold cross-validation on the validation set and was conducted in two steps: first, optimizing the V_{G} subset within 0-3 V range (FIGS. 21A-21C), and second, optimizing the time window within 14-343s range (FIGS. 21D-21F). In each step, the optimal model was selected based on the MSE and R² values between the predicted and ground truth cholesterol concentrations for 17 samples from the validation dataset. Subsets with a smaller V_{G} window were prioritized to minimize potential inter-batch variations for testing results from different testing days. Consequently, at the first step, the optimal V_{G} operating range was determined to be between 1.15 V and 2.45 V centered at 1.8 V (FIG. 21B). The predictions generated by the model with the optimal V_{G} window exhibited a strong correlation with the ground truth with an R² of 90.7%, however a CV of 20.7% was still high (FIG. 21C). To further enhance the accuracy, the optimal time range for a fixed optimal V_{G} window was determined by evaluating MSE and R² maps generated on the same validation dataset with 17 samples (FIG. 21D). The optimal time range based on lower MSE and higher R² was selected between 91 s and 119 s centered at 105 s, reducing the overall assay operation to <2.5 minutes (FIG. 21E). The predictions of the model with optimized V_{G} and time subsets on the validation set showed R² of 95.4% and a CV of 11.4% with respect to ground truth measurements (FIG. 21F), and the model with this input subset was further used during the blind testing stage.

While embodiments of the present invention have been shown and described, various modifications may be made without departing from the scope of the present invention. The invention, therefore, should not be limited, except to the following claims, and their equivalents.

1. A biosensor device comprising:
a cartridge having a chamber for receiving a fluid sample;
a porous sensing membrane disposed in the chamber and comprising one or more dried enzymes, enzyme-labeled antibodies, surfactants, substrates, stabilizers, buffers, or reagents disposed on or in the porous sensing membrane; and
a working electrode disposed in the cartridge and spaced apart from a surface of the porous sensing membrane.

2. The biosensor device of claim 1, wherein the porous sensing membrane comprises pores having effective diameters within the range of 20 nm to 20 μm.

3. The biosensor device of claim 1, wherein the working electrode is spaced apart from the surface of the porous sensing membrane at a distance within the range of 10 μm to 100 μm.

4. The biosensor device of claim 1, wherein the porous sensing membrane comprises polysulfone (PSF), polyestersulfone (PES), regenerated cellulose, or nitrocellulose (NC).

5. The biosensor device of claim 1, wherein the working electrode comprises indium tin oxide (ITO), tin oxide, zinc oxide, a metal oxide semiconductor, graphene, reduced graphene oxide, a conductive polymer, or a conductive 2D nanomaterial.

6. The biosensor device of claim 1, further comprising a field-effect transistor (FET) having a source, drain, and gate, wherein the gate is electrically coupled to the working electrode.

7. The biosensor device of claim 6, further comprising a reference electrode configured to contact with the fluid sample in the cartridge.

8. The biosensor device of claim 7, further comprising a semiconductor parameter analyzer or current-voltage circuitry coupled to the reference electrode and the FET.

9. A biosensor device comprising:
a cartridge having a chamber for receiving a fluid sample;
a plurality of porous sensing membranes disposed in the chamber, each porous sensing membrane comprising one or more dried enzymes, enzyme-labeled antibodies, surfactants, substrates, stabilizers, buffers, or reagents disposed on or in the respective porous sensing membrane; and
a plurality of working electrodes disposed in the cartridge and spaced apart from respective surfaces of the plurality of porous sensing membrane.

10. The biosensor device of claim 9, wherein the plurality of porous sensing membrane and the plurality of working electrodes are stacked vertically within the cartridge.

11. The biosensor device of claim 9, wherein the plurality of porous sensing membrane and their respective working electrodes spaced apart therefrom are configured to detect different biomarkers or analytes.

12. The biosensor device of claim 9, wherein each porous sensing membrane comprises pores having effective diameters within the range of 20 nm to 20 μm.

13. The biosensor device of claim 10, wherein each porous sensing membrane and its respective spaced apart working electrode are separated from each other at a distance within the range of 10 μm to 50 μm.

14. The biosensor device of claim 9, wherein the plurality of porous sensing membranes comprise polysulfone (PSF), polyestersulfone (PES), regenerated cellulose, or nitrocellulose (NC).

15. The biosensor device of claim 9, wherein the plurality of working electrodes comprise indium tin oxide (ITO), tin oxide, a metal oxide semiconductor, zinc oxide, graphene, reduced graphene oxide, a conductive polymer, or a conductive 2D nanomaterial.

16. The biosensor device of claim 9, further comprising a plurality of field-effect transistors (FETs) having a source, drain, and gate, wherein the respective gate of each FET is electrically coupled to different working electrodes of the plurality of working electrodes.

17. The biosensor device of claim 16, further comprising a reference electrode configured to contact the fluid sample in the cartridge.

18. The biosensor device of claim 17, further comprising a semiconductor parameter analyzer or current-voltage circuitry coupled to the reference electrode and the plurality of FETs.

19. A system for the detection and/or analysis of a biomarker or analyte in a fluid sample comprising:
a cartridge having a chamber for receiving the fluid sample, the cartridge comprising one or more porous sensing membrane disposed in the chamber, the one or more porous sensing membrane comprising one or more dried enzymes, enzyme-labeled antibodies, surfactants, substrates, stabilizers, buffers, or reagents disposed on or in the one or more porous sensing membrane, the cartridge further comprising one or more working electrodes disposed in the cartridge and spaced apart from respective porous sensing membrane of the one or more porous sensing membrane;
a reference electrode configured to contact with the fluid sample in the chamber;
one or more field-effect transistors (FETs) having a source, drain, and gate, wherein the respective gates of the one or more FETs is/are electrically coupled to the one or more working electrodes; and
a semiconductor parameter analyzer or current-voltage circuitry coupled to the reference electrode and the one or more FETs.

20. The system of claim 19, wherein the semiconductor parameter analyzer or current-voltage circuitry is configured to measure a change in initial threshold voltage (Vₜₕ) or drain current at constant specific gate voltage for the one or more FETs.

21. The system of claim 19, further comprising the semiconductor parameter analyzer or current-voltage circuitry is configured to generate and output a concentration of the biomarker or analyte based on the change in initial threshold voltage (Vₜₕ) or drain current at constant specific gate voltage for the one or more FETs.

22. The system of claim 19, wherein the semiconductor parameter analyzer or current-voltage circuitry generates FET transfer curves for the one or more FETs and the system further comprises a trained neural network that receives a data representation of the FET transfer curves and outputs a concentration of the biomarker or analyte.

23. A method for the detection and/or analysis of a biomarker or analyte in a fluid sample comprising:
providing a cartridge having a chamber for receiving the fluid sample, the cartridge comprising one or more porous sensing membrane disposed in the chamber, the one or more porous sensing membrane comprising one or more dried enzymes, enzyme-labeled antibodies, surfactants, substrates, stabilizers, buffers, or reagents disposed on or in the one or more porous sensing membrane, the cartridge further comprising one or more working electrodes disposed in the cartridge and spaced apart from the one or more porous sensing membrane;
providing one or more field-effect transistors (FETs) electrically coupled at respective gate(s) to the one or more working electrodes;
loading the fluid sample containing the biomarker or analyte into the cartridge;
measuring a change in initial threshold voltage (Vₜₕ) for the one or more FETs with a semiconductor parameter analyzer or current-voltage circuitry; and
outputting a concentration of the biomarker or analyte based at least in part on the change in initial threshold voltage (Vₜₕ) for the one or more FETs.

24. The method of claim 23, wherein measuring a change in initial threshold voltage (Vₜₕ) for the one or more FETs comprises recording FET transfer curves for the one or more FETs and wherein the FET transfer curves are converted to a two-dimensional signal heatmap.

25. The method of claim 24, further comprising inputting a data representation of the two-dimensional signal heatmap to trained neural network that outputs a concentration of the biomarker or analyte.

26. The method of claim 23, wherein the biomarker or analyte comprises cholesterol, low-density lipoprotein cholesterol (LDL-C) and/or high-density lipoprotein cholesterol (HDL-C).

27. (canceled)

28. The method of claim 23, wherein the biomarker or analyte comprises thyroid-stimulating hormone (TSH), glucose, triglyceride, creatinine, albumin, blood urea nitrogen, glycated hemoglobin, sodium, potassium, calcium, chlorine, phosphate, carbon dioxide, Alanine transaminase (ALT), Aspartate transaminase (AST), Alkaline phosphatase (ALP), bilirubin, Gamma-glutamyltransferase (GGT), progesterone, estrogen, estradiol, luteinizing hormone, or Anti-Mullerian hormone (AMH), COVID-19, influenza, HIV biomarkers, cancer biomarkers, disease biomarkers, or other pathogens.
